SEQUENCE LISTING (15 sequences)

SEQ ID NO: 1 - RNA, 22 nt, Artificial Sequence
  Synthetic miRNA probe
augcugacauauuuacuagagg

SEQ ID NO: 2 - RNA, 22 nt, Artificial Sequence
  Synthetic miRNA probe
ucggauccgucugagcuuggcu

SEQ ID NO: 3 - RNA, 21 nt, Artificial Sequence
  Synthetic miRNA probe
uucacaguggcuaaguuccgc

SEQ ID NO: 4 - RNA, 22 nt, Artificial Sequence
  Synthetic miRNA probe
uuuuucauuauugcuccugacc

SEQ ID NO: 5 - RNA, 23 nt, Artificial Sequence
  Synthetic miRNA probe
ugaggcucuguuagccuuggcuc

SEQ ID NO: 6 - RNA, 22 nt, Artificial Sequence
  Synthetic miRNA probe
uguaaacauccuugacuggaag

SEQ ID NO: 7 - RNA, 22 nt, Artificial Sequence
  Synthetic miRNA probe
aaggagcucacagucuauugag

SEQ ID NO: 8 - RNA, 23 nt, Artificial Sequence
  Synthetic miRNA probe
caacggaaucccaaaagcagcug

SEQ ID NO: 9 - RNA, 21 nt, Artificial Sequence
  Synthetic miRNA probe
aucacauugccagggauuucc

SEQ ID NO: 10 - RNA, 24 nt, Artificial Sequence
  Synthetic miRNA probe
aacaacaaaaucacuagucuucca

SEQ ID NO: 11 - RNA, 21 nt, Artificial Sequence
  Synthetic miRNA probe
uugugcuugaucuaaccaugu

SEQ ID NO: 12 - RNA, 23 nt, Artificial Sequence
  Synthetic miRNA probe
uggaagacuagugauuuuguugu

SEQ ID NO: 13 - RNA, 22 nt, Artificial Sequence
  Synthetic miRNA probe
uauugcacauuacuaaguugca

SEQ ID NO: 14 - RNA, 21 nt, Artificial Sequence
  Synthetic miRNA probe
uaccacaggguagaaccacgg

SEQ ID NO: 15 - RNA, 22 nt, Artificial Sequence
  Complementary synthetic RNA sequence to SEQ ID NO 1
uacgacuguauaaaugaucucc

(19) United States
(12) Patent Application Publication
(10) Pub. No.: US 2018/0171406 A1
(43) Pub. Date: Jun. 21, 2018

(54) IDENTIFICATION OF EPIGENETIC BIOMARKERS IN THE SALIVA OF CHILDREN WITH AUTISM SPECTRUM DISORDER

(71) Applicant: The Research Foundation for the State University of New York, Syracuse, NY (US)

(72) Inventors: Steven D. HICKS, Syracuse, NY (US); Frank A. MIDDLETON, Syracuse, NY (US)

(73) Assignee: The Research Foundation for the State University of New York, Syracuse, NY (US)

(21) Appl. No.: 15/545,093

(22) PCT Filed: Jan. 20, 2016
(86) PCT No.: PCT/US2016/014182

§ 371 Date: Jul. 20, 2017

Related U.S. Application Data
(60) Provisional application No. 62/106,107, filed on Jan. 21, 2015.
(60) Provisional application No. 62/263,467, filed on Dec. 4, 2015.

(51) Int. Cl. C12Q 1/6883; C12N 15/11

(52) U.S. Cl. CPC C12Q 1/6883; C12Q 1/686; C12N 2310/141; C12N 15/111

(57) ABSTRACT

The present invention relates to systems, devices, collections, techniques, and methods for diagnosing autism spectrum disorder (ASD) in a subject in addition to methods of treatment. In particular, the present disclosure relates to systems, devices, collections, techniques, and methods that utilize direct sequencing of micro ribonucleic acid (miRNA) or the use of miRNA probes having ribonucleotide sequences that may undergo nucleic acid hybridization with complementary nucleic acids present in a biological sample from the subject. Hybridization of the miRNA probes may be detected in the sample to determine if the subject has an ASD. Upon detection of an ASD using the methods discussed herein, therapy guidance may be effectuated to the subject.

### CROSS-REFERENCE TO RELATED APPLICATIONS

This application claims the benefit under 35 U.S.C. § 119(e) of U.S. Provisional Patent Application Nos. 62/263,467, filed Dec. 4, 2015, and 62/106,107, filed Jan. 21, 2015, which are incorporated by reference herein in their respective entireties.

### FIELD OF THE INVENTION

The present invention relates to systems, devices, collections, techniques, and methods for diagnosing autism spectrum disorder in a subject in addition to methods of treatment. In particular, the present disclosure relates to systems, devices, collections, techniques, and methods that utilize direct sequencing of micro ribonucleic acid (miRNA) or the use of miRNA probes having ribonucleotide sequences that may undergo nucleic acid hybridization with complementary nucleic acids present in a biological sample from the subject. Hybridization of the miRNA probes may be detected in the sample to determine if the subject has an autism spectrum disorder. Upon detection of an autism spectrum disorder using the methods discussed herein, therapy guidance may be effectuated to the subject.

### BACKGROUND OF THE INVENTION

Autism spectrum disorder (ASD) is a continuum of neurodevelopmental characteristics and/or disorders that is characterized by symptoms such as persistent deficits in social communication and social interaction and restricted repetitive patterns of behavior. These symptoms may be present in early child development, i.e., during the first two years of life, and may cause clinically significant impairment in social, occupational, or other important areas of life. According to a Center for Disease Control and Prevention survey of health and school records of 8-year-olds in 2010, ASD affects around 1 in 68 children in the U.S. Despite the prevalence of ASD, ASD lacks adequate screening tools, often delaying diagnosis and therapeutic interventions.

Pediatricians have an opportunity to improve outcomes for children with ASD through early diagnosis and referral for evidence-based behavioral therapy. Unfortunately, the first sign of ASD commonly recognized by pediatricians is a deficit in communication and language that does not manifest until 18-24 months of age. Indeed, early detection of ASD is important as early therapeutic intervention may significantly improve patient outcomes later in life.

Current screening tools for ASD in this age group include the Infant Toddler Checklist (ITC; also known as the Communication and Symbolic Behavior Scales and Developmental Profile) and the Modified Checklist for Autism in Toddlers (M-CHAT). The ITC may be used to identify developmental deficits in children ages 9-24 months, but has limited utility in distinguishing basic communication delays from overt ASD. The M-CHAT may be employed between 16 and 30 months. However, it requires a follow-up questionnaire for positive screens, which occur in 10% of children. Thus, the mean age of diagnosis for children with ASD is about 3 years, and approximately half of these may be false-positives.

ASD has been shown to include a substantial genetic component, with nearly 2,000 individual genes implicated in ASD. However, even though ASD includes this substantial genetic component, no single gene variant is specific to the disorder. Specifically, no single gene variant accounts for greater than 1% of ASD incidence, thus increasing the difficulty of screening for the disease.

Biomarker screening, which can be performed any time after birth, represents an attractive addition to the ASD screening toolkit. As stated above, a significant genetic component exists in ASD. ASD concordance rates are 50-90% among monozygotic twins compared with 0-30% among dizygotic twins, while full siblings have a two-fold greater concordance rate than half siblings. These figures suggest that ASD heritability could be as great as 50%. Potential transmission modes include copy number variation, single nucleotide variants, and single gene deletions.

An alternative mechanism for ASD pathogenesis includes epigenetic regulation/mechanisms, and these epigenetic mechanisms, including miRNAs, may contribute to the ASD phenotype by altering networks of neurodevelopmental genes. Extracellular transport of miRNA (through exosomes and other microvesicles) is an established epigenetic mechanism by which cells can alter their own gene expression and the expression of genes in cells around them. For the cells to alter the expression of genes around them, vesicular miRNA is extruded into the extracellular space, and this miRNA docks and enters neighboring cells to blocks translation of mRNA into proteins.

Thus, there exists a need for an improved screening tool to accurately diagnose ASD, preferably early in childhood. Additionally, there exists a need for a screening tool that can detect ASD painlessly and noninvasively.

### SUMMARY OF THE INVENTION

In an embodiment, a collection of 2 or more miRNA probes of a probe set may include ribonucleotide sequences selected from AUGCUGACAUAUUUACUAGAGG, UCGGAUCCGUCUGAGCUUGGCU, UUCACAGUGGCUAAGUUCCGC, UUUUUCAUUAUUGCUCCUGACC, UGAGGCUCUGUUAGCCUUGGCUC, UGUAAACAUCCUUGACUGGAAG, AAGGAGCUCACAGUCUAUUGAG, CAACGGAAUCCCAAAAGCAGCUG, AUCACAUUGCCAGGGAUUUCC, AACAACAAAAUCACUAGUCUUCCA, UUGUGCUUGAUCUAACCAUGU, UGGAAGACUAGUGAUUUUGUUGU, UAUUGCACAUUACUAAGUUGCA, or UACCACAGGGUAGAACCACGG (SEQ ID NOS. 1-14, respectively). In an implementation, the collection may include a relative ratio of 1 or more of the 14 miRNA probes to miRNAs present in a biological sample. The relative ratio may be between 1.5:1 and 2.5:1. In an implementation, a miRNA microarray may include a solid support and the collection of 2 or more miRNA probes may be attached to the solid support. The solid support may be a swab, saliva collection vial, or an assay plate.

In an embodiment, a method of diagnosing whether a subject has an autism spectrum disorder includes obtaining a biological sample from a subject potentially having an autism spectrum disorder; providing a collection of 1 or more miRNA probes of a probe set having a ribonucleotide sequence selected from SEQ ID NOS. 1-14; contacting the biological sample from the subject with the collection under conditions effective to permit hybridization of the probes to complementary nucleic acid molecules, if present, in the biological sample; detecting any hybridization as a result of contacting the biological sample with the collection; and identifying whether the subject has an autism spectrum disorder based on whether hybridization of the probes and nucleic acid molecules in the biological sample.

In an implementation, the method includes effectuating a therapy guidance if the subject has an autism spectrum disorder. The therapy guidance may include one or more of the following: performing additional diagnostic testing, prescribing a drug therapy, increasing monitoring frequency of the subject's autism spectrum disorder, recommending behavioral therapy and lifestyle choices, or a combination thereof. The therapy guidance may include recommending lifestyle choices such as: introducing behavioral techniques, managing time and task organization, introducing environmental changes, changes in diet, changes in exercise, or a combination thereof.

In an implementation, the collection may include relative ratios of 1 or more of the 14 miRNA probes to other miRNAs present in the biological sample. The relative ratios may be between 1.5:1 and 2.5:1. In an implementation, the biological sample may be saliva, urine, stool, serum, plasma, brain tissue, cerebrospinal fluid, and/or blood. In an implementation, detecting hybridization may be carried out by quantitative hybridization-based assays, including microarrays, Luminex-based magnetic or non-magnetic beads, Northern blot, RNase-Protection Assay, in situ hybridization (ISH), RNA-Scope and SMART Flare. In another implementation, detecting hybridization may determine miRNA expression level. In yet another implementation, detecting hybridization may be carried out by a polymerase chain reaction assay, including real-time quantitative PCR with oligonucleotide probes, SybrGreen or similar dye, radioactive nucleotide-based quantification, or digital droplet PCR.

In an embodiment, a method of diagnosing whether a subject has an autism spectrum disorder includes obtaining a biological sample from a subject potentially having an autism spectrum disorder; subjecting the biological sample to a direct sequencing process to provide nucleotide sequence information for nucleic acid molecules in the biological sample; comparing the nucleotide sequence information for nucleic acid molecules in the biological sample to nucleotide sequences of a collection of 1 or more miRNA probes selected from SEQ ID NOS. 1-14, or UACCACAGGGUAGAACCACGG; determining whether any of the collection of 1 or more miRNA probes or their complements are present in the biological sample, and determining the level of expression of any of the collection of 1 or more miRNA probes or their complements and relative levels of expression to each other based on comparing nucleotide sequence information; and identifying whether the subject has an autism spectrum disorder based on determining whether any of the collection of 1 or more miRNA probes or their complements are present in the sample.

In an implementation, the method further includes effectuating a therapy guidance if the subject has an autism spectrum disorder. The therapy guidance may include one or more of the following: performing additional diagnostic testing, prescribing a drug therapy, increasing monitoring frequency of the subject's condition, recommending behavioral therapy and lifestyle choices, or a combination thereof. The therapy guidance may include recommending lifestyle choices such as one or more of the following: introducing behavioral techniques, managing time and task organization, introducing environmental changes, changes in diet, changes in exercise, or a combination thereof.

In an embodiment, a kit suitable for determining whether a subject has an autism spectrum disorder is provided including 2 or more miRNA probes of a probe set having ribonucleotide sequences selected from SEQ ID NOS. 1-14. In an implementation, the kit further may include a solid support attached to the 2 or more miRNA probes. In an implementation, the kit may further include at least one of the following: (a) one randomly-generated miRNA sequence adapted to be used as a negative control; (b) at least one oligonucleotide sequence derived from a housekeeping gene, used as a standardized control for total RNA degradation; or (c) at least one randomly-generated sequence used as a positive control.

In an embodiment, a method of treating autism spectrum disorder in a subject includes obtaining a biological sample from the subject; providing a collection of 1 or more miRNA probes of a probe set having a ribonucleotide sequence selected from SEQ ID NOS. 1-14, or UACCACAGGGUAGAACCACGG; contacting the biological sample with the collection under conditions effective to permit hybridization of said probes to complementary nucleic acid molecules, if present, in the biological sample; detecting any hybridization as a result of contacting the biological sample with the collection of miRNA probes; identifying whether the subject has an autism spectrum disorder based on detecting hybridization; and effectuating a therapy guidance based on identifying that the subject has an autism spectrum disorder.

In an embodiment, a method of treating autism spectrum disorder in a subject includes obtaining a biological sample from a subject potentially having an autism spectrum disorder; subjecting the biological sample to a direct sequencing process to provide nucleotide sequence information for nucleic acid molecules in the biological sample; comparing the nucleotide sequence information for nucleic acid molecules in the biological sample to nucleotide sequences of a collection of 1 or more miRNA probes selected from the probe set including SEQ ID NOS. 1-14; determining whether any of the collection of 1 or more miRNA probes or their complements are present in the biological sample, and determining of the level of expression of the collection of 1 or more miRNA probes and/or their complements and relative levels of expression to each other based on comparing nucleotide sequence information; identifying whether the subject has an autism spectrum disorder based on determining whether any of the collection of 1 or more miRNA probes or their complements are present in the biological sample; and effectuating a therapy guidance if the subject is found to have an autism spectrum disorder.

### BRIEF DESCRIPTION OF THE DRAWINGS

FIG. 1 shows a table of subject characteristics.

FIG. 2 shows a table of top-ranked variables distinguishing ASD from control subjects and their correlations with neurodevelopment measures.

FIG. 3 shows a table of miRNA sequences corresponding to the top-ranked variables distinguishing ASD subjects from control subjects.

FIG. 4A shows a hierarchical cluster analysis of the top 14 miRNAs.

FIG. 4B shows a Partial Least Squares Discriminant Analysis (PLS-DA) of the top 14 miRNAs.

FIG. 4C shows a ROC-AUC analysis of the training data set indicated a very high level of performance in the logistic regression classification test (100% sensitivity, 90% specificity, with an AUC of 0.97).

FIG. 5A shows a ROC-AUC analysis illustrating an overall ROC-AUC of 0.92 and mis-classification of 3 ASD and 4 controls.

FIG. 5B shows the classification of subjects plotted by probabilities from the MCCV, with incorrectly classified subjects identified by ID number.

FIG. 5C shows whisker box plots of the four most robustly changed miRNAs according to the Mann-Whitney test.

### DETAILED DESCRIPTION

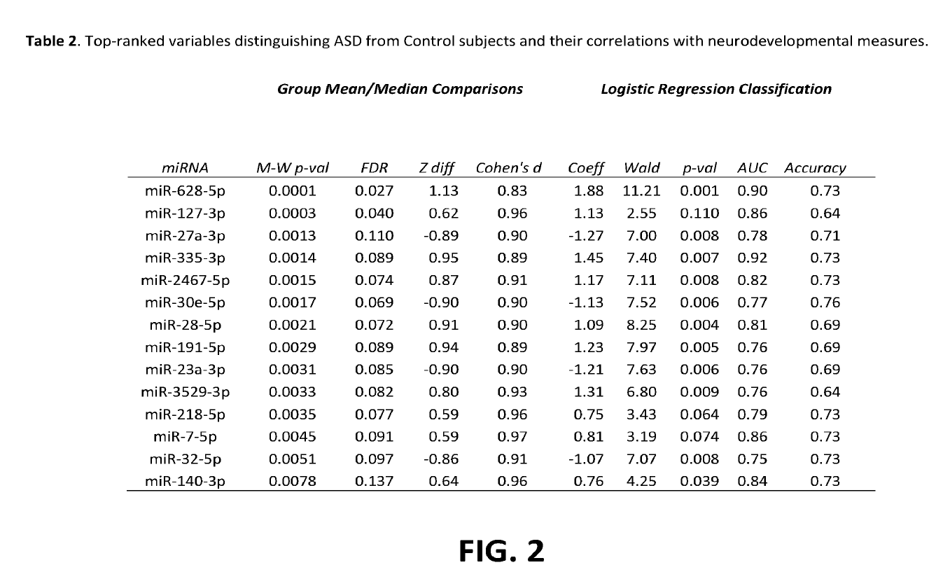

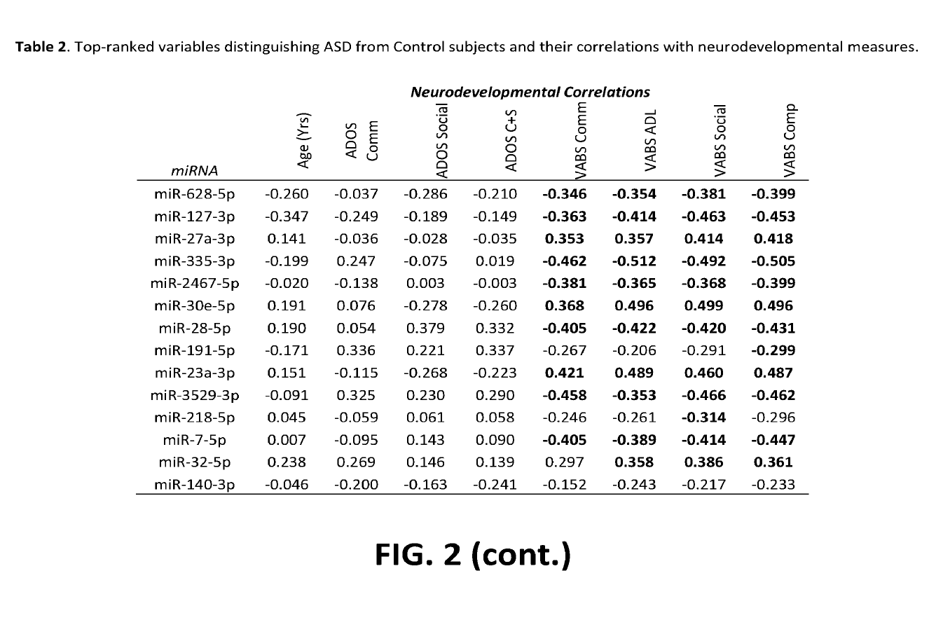

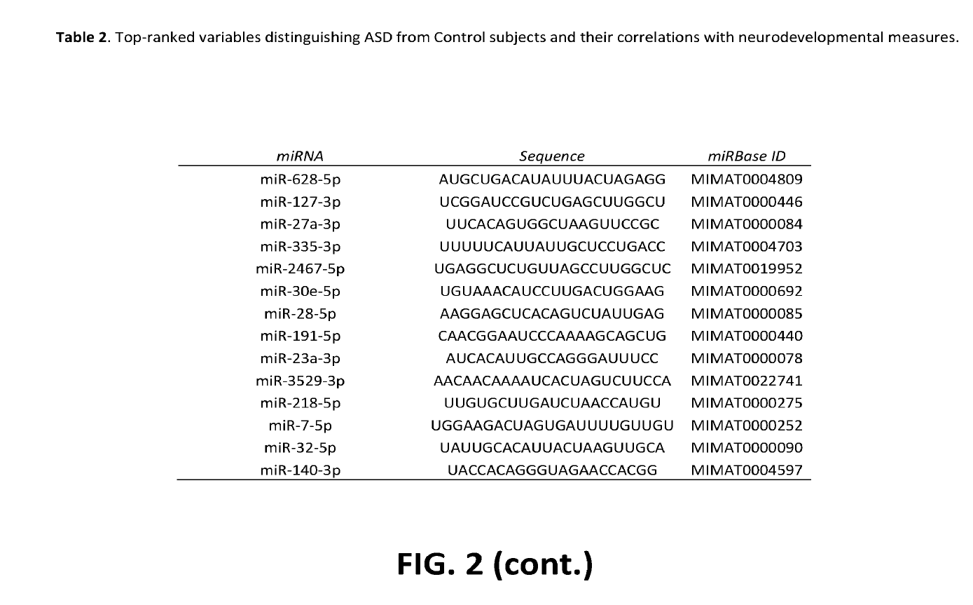

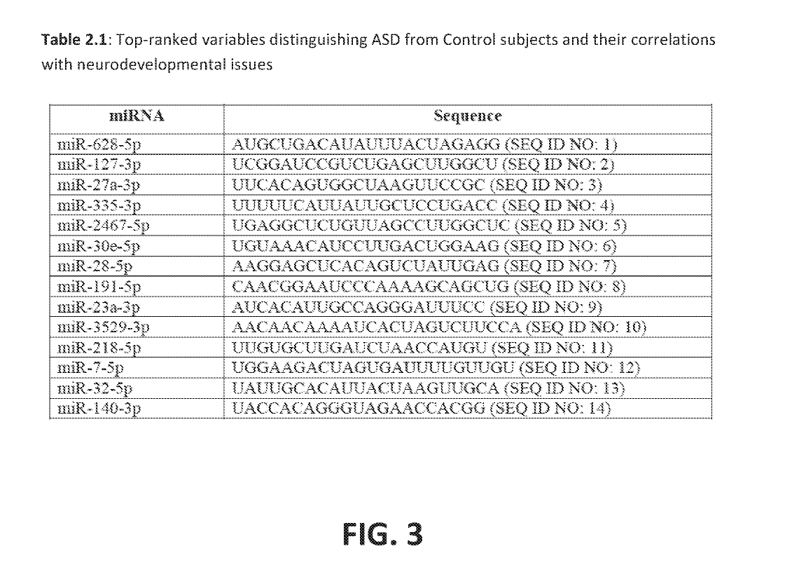

The systems, devices, collections, and methods of the present invention may generally be used to test a biological sample of a patient to determine whether that patient has an autism spectrum disorder. In particular, the present disclosure provides fourteen miRNAs listed in FIGS. 2 and 3 as sequence ID numbers (SEQ. ID NOS.) 1-14 that may comprise a probe set for detecting ASD. The miRNA sequences listed in FIGS. 2 and 3 corresponding to SEQ ID NOS 1-14 are (in order) AUGCUGACAUAUUUACUAGAGG, UCGGAUCCGUCUGAGCUUGGCU, UUCACAGUGGCUAAGUUCCGC, UUUUUCAUUAUUGCUCCUGACC, UGAGGCUCUGUUAGCCUUGGCUC, UGUAAACAUCCUUGACUGGAAG, AAGGAGCUCACAGUCUAUUGAG, CAACGGAAUCCCAAAAGCAGCUG, AUCACAUUGCCAGGGAUUUCC, AACAACAAAAUCACUAGUCUUCCA, UUGUGCUUGAUCUAACCAUGU, UGGAAGACUAGUGAUUUUGUUGU, UAUUGCACAUUACUAAGUUGCA, and UACCACAGGGUAGAACCACGG. Early testing suggests that these fourteen miRNAs may have a high correlation to ASD in children within the ages of 4 to 13 years. However, it will be understood that one of skill in the art will recognize that any suitable number of miRNAs having a high statistical correlation with an ASD may comprise a probe set for detecting ASD.

Because the extracellular nature of the epigenetic regulation mechanisms allows the measurement of genetic material from the central nervous system through simple collection of saliva, the present disclosure provides a method that minimizes many of the limitations of prior art methods, such as the limitations associated with analysis of post-mortem brain tissue (e.g., anoxic brain injury, RNA degradation, post-mortem interval, agonal state), or peripheral leukocytes (relevance of expression changes, painful blood draws). Thus, extracellular miRNA quantification in saliva provides an attractive and minimally invasive technique for biomarker identification in children with ASD. Accordingly, differential expression of brain-related miRNA may be detected in the saliva of ASD subjects, predictive of ASD classification, and related to neurodevelopmental measures of adaptive behavior.

Each of the miRNA probes may be adapted to undergo nucleic acid hybridization when in the presence of a complimentary ribonucleotide sequence in a biological sample under certain environmental conditions. Hybridization may be effectuated when environmental conditions such as, for example, temperature, pH, and/or duration of probe exposure is modified (i.e., increased or decreased). In one example, a probe having the first miRNA probe corresponding to miR-628-5p, AUGCUGACAUAUUUACUAGAGG, may be adapted to hybridize with its complimentary sequence, UACGACUGUAUAAAUGAUCUCC.

The complimentary ribonucleic acid sequences may correspond to ribonucleic sequences that have high statistical correlations with, or otherwise indicate, an autism spectrum disorder. Thus, by detecting the hybridized ribonucleic sequences in the biological sample, complimentary ribonucleic sequences that were present in the sample may be identified to ultimately identify the presence of an autism spectrum disorder in a subject.

In an exemplary method, a biological sample may be extracted (and in some embodiments, also purified through, e.g., RNA extraction) from a subject who potentially has an autism spectrum disorder. Non-limiting examples of biological samples include saliva, urine, stool, serum, plasma, brain tissue, cerebrospinal fluid and/or blood. The above listed biological samples may be preferable in some embodiments due to the extracellular availability of miRNAs therein that allows for painless, noninvasive collection.

In an example where saliva is collected as the biological sample, the purification process, if used, may include purifying salivary RNA in accordance with, for example, the Oragene RNA purification protocol using TRI Reagent LS, a TriZol purification method, or similar method. The Oragene purification protocol generally includes multiple parts. In the first part, a sample is shaken vigorously for 8 seconds or longer and the sample is incubated in the original vial at 50° C. for one hour in a water bath or for two hours in an air incubator. In the second part, a 250-500 μL aliquot of saliva is transferred to a microcentrifuge tube, the microcentrifuge tube is incubated at 90° C. for 15 minutes and cooled to room temperature, the microcentrifuge tube is incubated on ice for 10 minutes, the saliva sample is centrifuged at maximum speed (>13,000×g) for 3 minutes, the clear supernatant is transferred into a fresh microcentrifuge tube and the precipitate is discarded, two volumes of cold 95% EtOH is added to the clear supernatant and mixed, the supernatant mixture is incubated at −20° C. for 30 minutes, the microcentrifuge tube is centrifuged at maximum speed, the precipitate is collected while the supernatant is discarded, the precipitate is dissolved in 350 μL of buffer RLT, and 350 μL of 70% EtOH is added to the dissolved pellet mixture and mixed by vortexing. The first two parts may be followed by the Qiagen RNeasy cleanup procedure.

The purification process may further include a second purification step of, for example, purifying the saliva sample using a RNeasy mini spin column by Qiagen. One of skill in the art will recognize that purification of the biological sample may include any suitable number of steps in any suitable order. Purification processes may also differ based on the type of biological sample collected from the subject. The yield and quality of the purified biological sample may be assessed via a device such as an Agilent Bioanalyzer, for example, to determine if the yield and quality of RNA is above a predetermined threshold.

After the biological sample has been purified, a collection of one or more miRNA probes may be provided from the probe set. As stated above, the probe set may include the ribonucleotide sequences listed in FIGS. 2 and 3. Thus, the collection may comprise a subset of miRNA probes from the probe set, or all of the miRNA probes from the probe set. After consideration of this disclosure, one of skill in the art will recognize that the collection may comprise any suitable number of miRNA probes from the probe set, such as, for example, 1 miRNA probe, 2 miRNA probes, 3 miRNA probes, 4 miRNA probes, 5 miRNA probes, 6 miRNA probes, 7 miRNA probes, 8 miRNA probes, 9 miRNA probes, 10 miRNA probes, 11 miRNA probes, 12 miRNA probes, 13 miRNA probes, or 14 miRNA probes.

In an embodiment of the invention, the collection may comprise a relative ratio of one or more of the miRNA probes described above to complimentary miRNAs present in the biological sample. In an example, a 1:1, 1.5:1, 1:2, 1:3, 2.5:1, 2:3, 1:4, or 1:5 ratio may be used, although one of skill in the art will recognize that any suitable ratio of a particular miRNA to complimentary miRNAs in the biological sample may be used.

The miRNA probe collection may be applied to (or contacted with) the purified sample to effect nucleic acid hybridization under certain environmental conditions, e.g., contact between the miRNA probe and a complementary nucleic acid in an environment of controlled temperature and pH. After contacting the probe collection to the purified sample, any hybridization may be detected by performing, for example, a polymerase chain reaction (PCR) assay. In another embodiment, detection of hybridization may also include real-time quantitative reverse-transcription (RT) PCR with oligonucleotide primers or probes, SybrGreen, FAM, ROX, or a similar fluorescent dye, radioactive nucleotide-based quantification, and/or digital droplet quantitative PCR.

In another embodiment, detection and/or measurement of miRNA levels can be performed by a large variety of techniques, including but not limited to: direct sequencing of the biological sample using a sequencer; microarrays with nucleotide probes such as the miRNA probes described above; Luminex-based magnetic and/or non-magnetic beads with nucleotide probes; PCR with nucleotide probes and/or custom primers; RNAse Protection Assays with custom protection probes; Northern blot with nucleotide based probes; RNAscope and Smart Flare with custom hybridization probes; and radioactive nucleotide incorporation run-off and PCR-based quantification.

If the biological sample is subjected to a direct sequencing process (e.g., shotgun sequencing or direct sequencing) to provide nucleotide sequence information for nucleic acid molecules in the biological sample, the nucleotide sequencing information for nucleic acid molecules in the biological sample may be compared to known nucleotide sequences, such as, for example, one or more nucleotide sequences in FIGS. 2 and 3 having SEQ ID NOS. 1-14. If one or more of the above mention nucleotide sequences (or their complements) are detected, levels of expression of the nucleotide sequences and/or relative levels of expression to one another within the biological sample may be determined. Levels of expression may be determined through “read counts” (matched sequences with known miRNA sequences) that are either normalized or reported as ratios between the matched sequences and the known sequences. Autism spectrum disorder may be identified in the subject based on the detection of one or more of the nucleotide sequences (or their complements). Additionally, the levels of expression of the nucleotide sequences and/or the relative levels of expression of the nucleotide sequences to one another may be used to identify an autism spectrum disorder in the subject.

The present disclosure also provides for a miRNA microarray for detecting autism spectrum disorder. The miRNA microarray may include a solid support, such as a swab, for example. In another embodiment, the solid support may be an assay plate or a saliva collection vial. The solid support may comprise one or more of the miRNAs from the probe set as described above attached thereon.

The present disclosure further provides for a kit for determining whether a subject has an autism spectrum disorder. The kit may include 2 or more miRNA probes of the probe set having ribonucleotide sequences selected from the group comprising the following miRNA sequences (corresponding to SEQ ID NOS. 1-14) as shown in FIGS. 2 and 3: AUGCUGACAUAUUUACUAGAGG, UCGGAUCCGUCUGAGCUUGGCU, UUCACAGUGGCUAAGUUCCGC, UUUUUCAUUAUUGCUCCUGACC, UGAGGCUCUGUUAGCCUUGGCUC, UGUAAACAUCCUUGACUGGAAG, AAGGAGCUCACAGUCUAUUGAG, CAACGGAAUCCCAAAAGCAGCUG, AUCACAUUGCCAGGGAUUUCC, AACAACAAAAUCACUAGUCUUCCA, UUGUGCUUGAUCUAACCAUGU, UGGAAGACUAGUGAUUUUGUUGU, UAUUGCACAUUACUAAGUUGCA, or UACCACAGGGUAGAACCACGG. The kit may further include at least one of the following: (a) one randomly-generated miRNA sequence adapted to be used as a negative control; (b) at least one oligonucleotide sequence derived from a housekeeping gene, to be used as a standardized control for total RNA degradation; and/or (c) at least one randomly-generated sequence used as a positive control. Non-limiting examples of housekeeping genes include lactate dehydrogenase A (LDHA), Non-POU domain-containing octamer-binding protein (NONO), phosphoglycerate kinase 1 (PGK1), and/or peptudyl-prolyl cis-trans isomerase H (PPIH).

Upon detection of an autism spectrum disorder in a subject, therapy guidance may be provided to the subject. Such guidance may include: performing additional diagnostic testing, prescribing a drug therapy, increasing monitoring frequency of the subject's autism spectrum disorder, recommending behavioral therapy and lifestyle choices, or a combination thereof. The therapy guidance may include recommending lifestyle choices such as: introducing behavioral techniques, managing time and task organization, introducing environmental changes, changes in diet, changes in exercise, or a combination thereof. In some embodiments, the therapy guidance may alternatively or additionally include one or more of the following: performing additional diagnostic testing, prescribing a drug therapy, increasing monitoring frequency of the subject's autism spectrum disorder, recommending behavioral therapy and lifestyle choices, or a combination thereof.

In another embodiment, sequence libraries may be prepared from the purified biological sample using the Illumina TruSeq Small RNA Sample Prep protocol.

After the sequence libraries are prepared for the purified biological sample, the sequence libraries may be processed through a high throughput sequencing device, such as an Illumina MiSeq, NextSeq, or HiSeq using, for example, the most recent reagents at a target depth of 3 to 10 million reads per sample. The read data may be aligned to a reference genome (such as the Hg19 Build of the human genome, for example) and cross-referenced to the most current miRNA database or a reference miRNA, such as those listed in FIGS. 2 and 3, using a processor and software adapted to process the read data of the human genome and compare the human genome read data to the reference genome or reference miRNA, such as the Illumina BaseSpace software, for example. As used herein, a reference genome is a nucleic acid sequence database that serves as a representative example of a species set of genes. Reference genomes may be assembled from a number of donors so that they are not biased by the influence of genetics from a single person (to form a haploid mosaic). Build 21 represents one collection of sequences derived from healthy volunteers, although the present disclosure contemplates using other suitable reference genomes. Sequence aligning and/or analysis algorithms such as, for example, the Bowtie sequence aligner may be used to further process the read data and the data may be normalized prior to further analysis.

After sequence aligning and/or analysis is performed, the subject may be diagnosed with an autism spectrum disorder based on the miRNAs that are detected in the sample or based on comparisons made to miRNA data in a sequencer. Therapy guidance may be provided to the subject based on the determination that the subject has an autism spectrum disorder. Examples of therapy guidance may include, but are not limited to: recommending lifestyle choices and includes one or more of the following: introducing behavioral techniques, managing time and task organization, introducing environmental changes, changes in diet, changes in exercise, performing additional diagnostic testing, prescribing a drug therapy, increasing monitoring frequency of the subject's autism spectrum disorder, recommending behavioral therapy and lifestyle choices, or a combination thereof

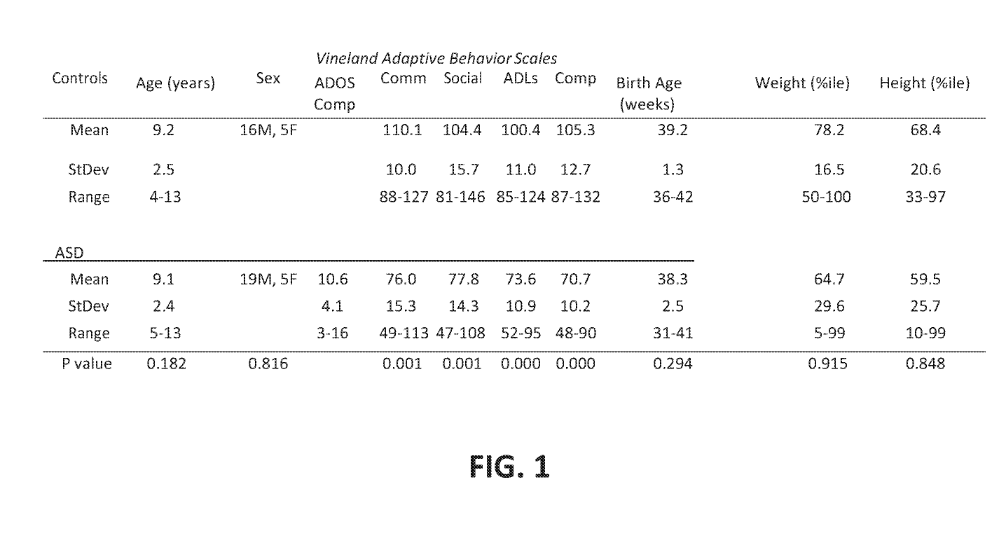

FIG. 1 shows a table of subject characteristics for the control subjects and the ASD subjects. As shown in FIG. 1, characteristics for both the control subjects and the ASD subjects include age (in years), sex, Autism Diagnostic Observation Schedule (ADOS) Score, Vineland Communication Score (Comm), Vineland Socialization Score (Social), Activities of Daily Living Score (ADLs), Composite Score (Comp), birth age (in weeks), weight (in percentile), and height (in percentile). A mean, standard deviation, and range is included for select characteristics in both the control group and the ASD group. P-values are also computed for select characteristics at the bottom of the table. As can be appreciated, Vineland scores including Comm, Social, ADLs, and Comp differ significantly between the control and ASD groups.

FIG. 2 shows a table of top-ranked variables distinguishing ASD from control subjects and their correlations with neurodevelopment measures. As shown in FIG. 2, the 14 top ranked variables (miRNAs) distinguishing ASD from control subjects are shown in addition to each sequence associated with the particular miRNA listed. FIG. 2 further includes values indicating the relative strength of neurodevelopment correlations associated with each listed miRNA.

FIG. 3 shows a table of miRNA sequences corresponding to the top-ranked variables distinguishing ASD subjects from control subjects. The sequences and miRNA listed in FIG. 3 correspond to those listed in FIG. 2.

Variations and modifications will occur to those of skill in the art after reviewing this disclosure. The disclosed features and steps may be implemented, in any combination and subcombination (including multiple dependent combinations and subcombinations), with one or more other features and/or steps described herein. The various features and steps described or illustrated above may be combined or integrated in other systems and methods. Moreover, certain features may be omitted or not implemented.

Examples of changes, substitutions, and alterations are ascertainable by one skilled in the art and could be made without departing from the scope of the invention disclosed herein. All references cited herein are incorporated by reference in their entirety and made part of this application.

Summary of Experiment:

Salivary miRNA profiles identify children with autism spectrum disorder, correlate with adaptive behavior, and implicate ASD candidate genes involved in neurodevelopment. In particular, salivary miRNA was purified from 24 ASD subjects and 21 age- and gender-matched control subjects. The ASD group included individuals with mild ASD (DSM-5 criteria and Autism Diagnostic Observation Schedule) and no history of neurologic disorder, pre-term birth, or known chromosomal abnormality. All subjects completed a thorough neurodevelopmental assessment with the Vineland Adaptive Behavior Scales at the time of saliva collection. A total of 246 miRNAs were detected and quantified in at least half the samples by RNA-Sequencing (RNA-Seq) and used to perform between-group comparisons with non-parametric testing, multivariate logistic regression and classification analyses, as well as Monte-Carlo Cross-Validation (MCCV). The top miRNAs were examined for correlations with measures of adaptive behavior. Functional enrichment analysis of the highest-confidence mRNA targets of the top differentially expressed miRNAs was performed using the Database for Annotation, Visualization, and Integrated Discovery (DAVID), as well as the Simons Foundation Autism Database (AutDB) of ASD candidate genes.

14 miRNAs were differentially expressed in ASD subjects compared to controls (p<0.05; FDR<0.15) and showed more than 95% accuracy at distinguishing subject groups in the best-fit logistic regression model from the discovery set. MCCV revealed an average ROC-AUC value of 0.92 across 100 simulations, further supporting the robustness of the findings. Most of the 14 miRNAs showed significant correlations with Vineland neurodevelopmental scores. Functional enrichment analysis detected significant over-representation of target gene clusters related to transcriptional activation, neuronal development, and AutDB genes.

Measurement of salivary miRNA in this pilot study of subjects with mild ASD demonstrated differential expression of 14 miRNAs that are expressed in the developing brain, impact mRNAs related brain development, and correlate with neurodevelopmental measures of adaptive behavior. These miRNAs have high specificity and cross-validated utility as a potential screening tool for ASD.

Description of Experiment

Subjects and Assessments

Subjects were recruited and the exclusion criteria for both control and ASD subjects included an age less than 4 years or greater than 14 years, confounding neurological (i.e. cerebral palsy, epilepsy) or sensory (i.e. auditory or visual impairment) disorders, or acute illness. Wards of the state, subjects with mental retardation or a history of pre-term birth (less than 32 weeks gestation) or birth weight less than 10^{th} percentile for gestational age were also excluded from participation. Subjects with a diagnosis of intellectual disability, ASD, or a family history of ASD were excluded from the control group. ASD subjects with a known syndromic phenotype (i.e. Rett Syndrome, Tuberous Sclerosis, Angelman Syndrome, Fragile X) were also excluded. Given the established comorbidity of psychiatric symptoms in children with ASD, subjects with attention deficit hyperactivity disorder (ADHD) or anxiety were not excluded.

Parental consent and subject assent (when possible) were obtained for a total of 45 subjects who were recruited for the study, including 24 subjects with a current diagnosis of ASD and 21 non-ASD control subjects (as shown in FIG. 1). ASD subjects were diagnosed according to DSM-5 (American Psychiatric Association, 2013) criteria and were evaluated with an age-appropriate module of the Autism Diagnostic Observation Schedule (ADOS), the Childhood Autism Rating Scale (CARS), and/or the Krug Asperger Index. The Vineland Adaptive Behavior Scales 2^{nd} edition was administered to all children by a physician through parental interview to evaluate functional neurodevelopmental indices of communication (Comm), social interaction (Social), and activities of daily living (ADLs). Medical history, birth history, family history, surgical history, current medications, medical allergies, immunization status, and dietary modifications were obtained. A brief physical exam was performed to screen for neurologic deficits, visual/hearing impairment, or syndromic physical features.

As shown in FIG. 1, there were no significant differences between groups in age (p=0.18), sex (p=0.82), weight (p=0.91), height (p=0.85), or birth age (p=0.29). The mean age of the ASD subjects was 9.2±2.5 years and the mean birth weight was 3.2±0.64 kg. The ASD subjects had a mean ADOS score of 10.6±4.1, consistent with DSM-5 criteria for mild to moderate ASD. Compared with control subjects they displayed significantly decreased levels of Communication (p<0.001), Social Interaction (p=0.001) and Activities of Daily Living (p<0.001) as assessed by Vineland Adaptive Behavior Scales (FIG. 1).

Overall, the ASD group of 24 children included several with comorbid diagnoses: ADHD (n=15), anxiety disorder (n=8), learning disability or developmental delay (n=5), asthma (n=3), allergies (n=2), obsessive-compulsive disorder (n=2), and depression (n=1). Reported medications in this group included: methylphenidate stimulants (n=8), serotonin specific reuptake inhibitors (SSRIs; n=7), guanfacine (n=5), atypical antipsychotics (n=5), clonidine (n=1), bronchodilators (n=3), anti-histamines (n=3), multivitamins (n=8) and omega-3 supplements (n=4). Three of the probands were eating a modified gluten-free diet and no ASD subjects had any dental carries or periodontal disease. Five ASD subjects had a history of birth complications requiring neonatal intensive care, although none required care beyond 11 days. Most (n=17) of the ASD subjects had a current or past history of educational intervention (speech therapy, physical therapy, occupational therapy). There were also several probands with positive family histories of neuropsychiatric and neurodevelopmental disorders (limited to 1st and 2nd degree relatives and 1st cousins): learning disability (n=10), depression (n=8), anxiety disorder (n=7), ADHD (n=6), ASD (n=4), and bipolar disorder (n=3).

The control group of 21 typically developing children also included several with comorbid diagnoses: ADHD or ADD (n=5), asthma (n=6), eczema (n=4), and allergies (n=2). Reported medications in the control group included: methylphenidate (n=3), bronchodilators (n=6), and antihistamines (n=5). None of the control children were eating a modified or gluten-free diet and one subject had dental carries. One control subject had a history of birth complication (RSV infection) that required a brief period of neonatal care. Three of the control subjects had a current or past history of educational intervention (speech therapy, physical therapy, occupational therapy). Positive family histories among 1st and 2nd degree relatives and 1st cousins were identified for learning disability (n=2), depression (n=1), ADHD (n=1), and bipolar disorder (n=1).

Saliva Collection and miRNA Processing

Subjects were recruited during well-child visits. Saliva samples were collected in a non-fasting state between 10 am and 3 pm. After rinsing with tap water, approximately 3 mLs of saliva were obtained via expectoration using an Oragene RNA collection kit (DNA Genotek; Ottawa, Canada) and stored at room temperature until processing. Salivary miRNA was purified according to the Oragene RNA purification protocol using TRI Reagent LS, followed by a second round of purification using the RNeasy mini column (Qiagen). The yield and quality of the RNA samples was assessed using the Agilent Bioanalyzer prior to library construction using the IIlumina TruSeq Small RNA Sample Prep protocol (IIlumina; San Diego, Calif.). Multiplexed samples were run on an IIlumina MiSeq instrument using v3 reagents at a targeted depth of 3 million reads per sample. Reads were aligned to Build 20 of the human genome in Illumina BaseSpace Software using the Bowtie algorithm and normalized to reads per million (RPM) prior to analysis. The data set supporting the results of this article is available in the NCBI Gene Expression Omnibus repository (accession number pending).

Statistical Analysis

Analysis of the combined medical, demographic, and neuropsychological data was performed to identify significant group differences between ASD and control subjects. Individual miRNAs were used for comparisons between groups only if they were detected in at least half the samples regardless of diagnosis. A total of 246 miRNAs were tested. Because the RNA-Seq data were not normally distributed, group differences in miRNA levels were examined using a non-parametric Wilcoxon Mann-Whitney U test with Benjamini-Hochberg False Discovery Rate (FDR) correction for multiple comparisons. The miRNAs with FDR values <0.15 were initially used in individual logistic regression analyses to assess discriminative power in an idealized “best-fit” approach. The rationale for doing so was the fact that logistic regression makes no assumption about the distribution of the original RNA-Seq data and it is highly effective at iteratively determining an optimal model for the data using the logistic function Y=[1/(1+e^{-(a+b}^{₁}^{X}^{₁}^{+b}^{₂}^{X}^{₂}^{+b}^{ₙ}^{X}^{ₙ}^{+ . . . )})] that best describes the dependency of the dependent outcome (diagnosis, coded as 0 or 1) on the full set of 14 independent variables. This best fitting was accomplished by adjustment of the partial regression coefficients for each miRNA variable until an optimal solution was obtained using the Maximum Likelihood criterion. During this process, each subject sample was determined to have a specific likelihood of falling in one of the diagnostic classes based on the model and the total likelihood (L) for the set of subjects was derived from the running product of the likelihood scores for all of the subjects. Since a prediction was made for each subject, the results of the logistic regression analysis were then used to produce a 2×2 classification table from which we determined the Sensitivity or True Positive Rate (i.e., fraction of ASD subjects who were correctly predicted to be ASD based on the model) and the Specificity or True Negative Rate (i.e., the fraction of Control subjects who were correctly predicted to be Controls). The cutoff points for the classification were set by default to be Y=0.5 (halfway between the diagnostic category coding of 0 and 1). By varying the cutoff point across the full range of cutoff values and recalculating the Sensitivity and Specificity at each point, it was then possible to construct a receiver operating characteristic (ROC) plot which provided an unbiased assessment of the overall model performance.

To facilitate comparisons with other data sets, mean differences in abundance seen in ASD subjects were reported as normalized Z score differences relative to controls as well as standardized Cohen's d values, which incorporate the variability within each subject group. We also reported the beta coefficients (Coeff) and Wald statistics with resulting p values for each of the individual regression results. Comparisons of miRNA levels to various medical, demographic and neuropsychological measures were performed using Spearman's rank correlation.

One of the limitations of any regression modeling approach is the possibility that the “best fit” only accurately predicts outcomes in the initial (discovery) data set. To more stringently evaluate the empirical validity of the 14 miRNAs, we performed classification testing and ROC curve analysis based on the results of 100-fold Monte-Carlo Cross Validation (MCCV) with balanced subsampling. In each iteration two-thirds of the samples were used to evaluate the miRNA feature importance. Next, the 2, 3, 5, 7, 10 and 14 most important classifying miRNA features were used to build classification models which were cross-validated on the remaining one-third of the samples that were left out. The procedure was repeated 100 times to determine the performance and confidence interval of each model. To further complement the logistic modeling we did in the discovery phase, this MCCV analysis was performed using the multivariate linear regression approach of Partial Least Squares Discriminant Analysis (PLS-DA). This method extracts multidimensional linear combinations of the 14 miRNA features that best predict the class membership or diagnosis (Y). These analyses were performed using the plsr function provided by R pls package, with classification and cross-validation performed using the caret package. We also ranked the variables by their relative importance, as determined by the sum of regression coefficients in the different simulated models, and generated individual boxplots for the 4 most robust differentially expressed miRNAs.

To visualize the expression patterns and general separation power of the set of significantly changed miRNAs, we used hierarchical clustering with a Euclidian distance metric to group miRNAs with similar patterns together, and then visualized the subjects in the 3 eigenvector dimensions created from the PLS-DA analysis of all 14 miRNAs.

Systems-level analysis of the miRNA data was performed using the miRNA Data Base (miRDB) online resource to provide the predicted targets for each of the mature sequences that we identified (according to mirBase v21 Aug. 2014 annotation). This database version identifies 2,588 human miRNAs and 947,941 target interactions. The interactions that were revealed were then filtered based on the predicted strength of the miRNA-mRNA interaction, as reflected in the miRDB output to include only the top 20% of predicted targets for each miRNA. These specific mRNAs were then examined for evidence of functional enrichment using the online Functional Annotation Clustering tool from the Database for Annotation, Visualization, and Integrated Discovery (DAVID, version 6.7) at the National Institute of Allergy and Infectious Diseases (NIAID). Because of the large number of genes being examined, we increased the EASE score threshold to 2.0, and set the Multiple Linkage Threshold to 0.7, the Similarity Threshold set to 0.45, and the Final Group Membership set size to 4. Only the top 3 Annotation Clusters were reported in table form. In addition to the DAVID functional clusters, we also compared the list of the top 20% of predicted targets for the combined set of 14 miRNAs to the 740 ASD-associated genes catalogued in the Simons Foundation Autism Database (AutDB) and tested for possible enrichment using a Fisher's Exact test and Odds Ratio calculation.

Brain and tissue-specific expression patterns for differentially expressed miRNAs were identified by review of a survey of differentially expressed miRNAs across the developing and adult human brain. We also used the brain data to note whether miRNAs that were highly-expressed in brain were also detected in the saliva regardless of whether they were altered in ASD.

Measurement of miRNA levels can be performed by a large variety of techniques, including direct sequencing using a sequencer, microarrays with nucleotide probes such as the miRNA probes described above, Luminex-based magnetic and non-magnetic beads with nucleotide probes, polymerase chain reaction (PCR) with nucleotide probes and/or custom primers, RNAse Protection Assays with custom protection probes, Northern blot with nucleotide based probes, RNAscope and Smart Flare with custom hybridization probes, and radioactive nucleotide incorporation run-off and PCR-based quantification.

Saliva miRNA Levels Show Relationship to Diagnostic and Adaptive Behavior Measures

Sequencing of salivary miRNAs detected 246 miRNAs as being present in at least half the samples. Among these, 14 miRNAs showed significant changes in expression according to a Mann-Whitney test (p<0.05, FDR<0.15) in the ASD group compared with controls (as shown in FIG. 2). Ten of the miRNAs were up-regulated in ASD subjects and four were down-regulated. The miRNA with the largest mean difference in abundance between ASD and control subjects was miR-628-5p (it also had the most significant difference) (p=0.0001, Z score difference=1.13). Results from the individual logistic regression analyses also highlighted miR-628-5p as the most significant (Wald statistic=11.21, p=0.001), and it showed the second highest area under the curve (AUC=0.90) from the ROC analysis (FIG. 2). Individually, miR-335-3p had the largest AUC and miR-30e-5p had the highest accuracy in predicting ASD diagnosis at 76% (FIG. 2).

To determine if the 14 miRNAs of interest were associated with neurodevelopmental measures, we performed Spearman's rank correlation analysis. This analysis revealed significant correlations between multiple measures included in the Vineland scores and 13 of the 14 miRNAs (only miR-140-3p failed to show significant correlation). Notably, 9 of the miRNAs demonstrated only negative correlations (i.e., higher miRNA was associated with lower Vineland scores) while 4 of them showed only positive correlations. Furthermore, every miRNA with positive correlations to Vineland scores was one that had reduced expression in ASD subjects, whereas every miRNA with negative correlations to Vineland scores had increased expression in ASD (FIG. 2).

FIG. 3 shows a table of miRNA sequences corresponding to the top-ranked variables distinguishing ASD subjects from control subjects. The sequences and miRNA listed in FIG. 3 relate to those listed in FIG. 2.

Hierarchical Clustering and Linear Discriminant Analysis Distinguish Samples by miRNA Levels

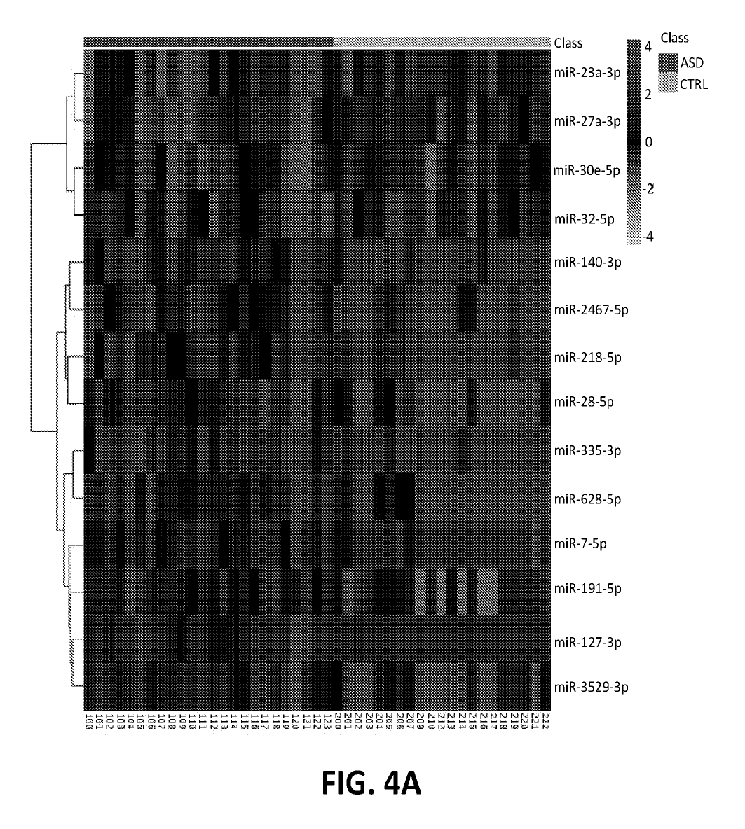

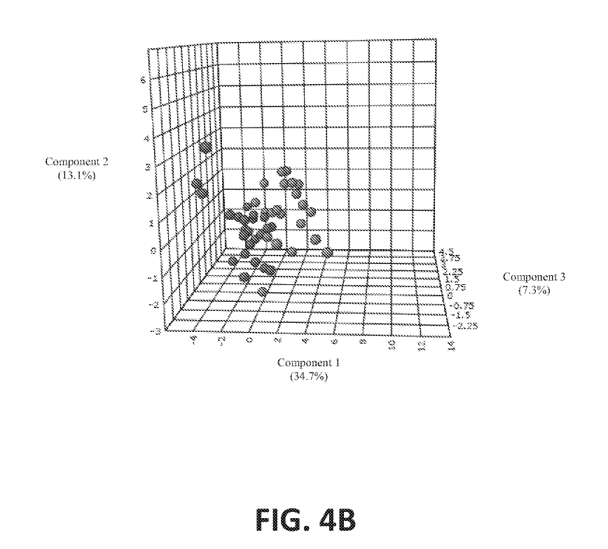

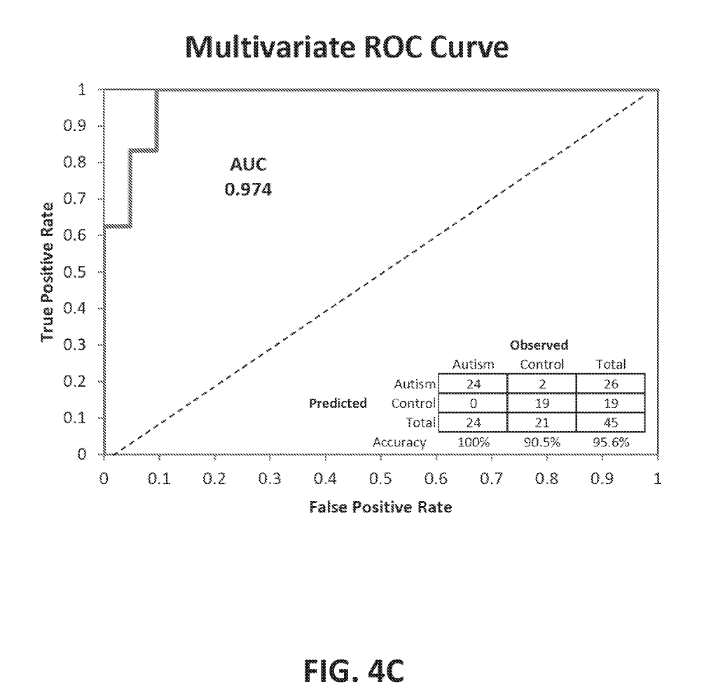

Hierarchical clustering was performed for ASD and Control subjects to reveal salient patterns in the miRNA data. (FIG. 4A). The PLS-DA results were used to visualize the degree of separation between ASD and control subjects using a three-dimensional representation of the 14 variable matrix. The results of this analysis complemented the clustering results and indicated only moderate overlap in the subject groups (FIG. 4B). Examination of the medical, demographic, and adaptive behavior data for these overlapping Control and ASD subjects in the 3-dimensional plots failed to identify any definitive explanations.

Multivariate Regression, Class Prediction, and ROC Analysis Indicate High Sensitivity and Specificity

The initial “best-fit” model to assess the maximal diagnostic utility was based on a single multivariate logistic regression test for classification accuracy. The results of this were evaluated using a Receiver Operating Characteristic (ROC) curve and classification prediction table (FIG. 4C). Multivariate ROC for this set of miRNAs revealed an area under the curve (AUC) of 0.974. This miRNA set was 100% sensitive and 95.6% specific for predicting the diagnosis of ASD within the study participants. Notably, because we pre-selected our subjects into either ASD or control groups, we did not determine the positive predictive value or negative predictive value of the 14 variables.

Cross-Validation of Diagnostic Utility of miRNA Data Set

The data set of 14 miRNAs variables continued to perform at a very high level in the Monte-Carlo Cross-Validation (MCCV) experiments, with an average ROC AUC value of 0.92 for the full model (containing all 14 miRNAs). Furthermore, the MCCV revealed 87.5% specificity and 81% sensitivity, with an overall accuracy of 84.4% across 100 simulations. The most common outcome was a confusion matrix that contained 4 misclassified Controls and 3 or 4 misclassified ASD subjects. Notably, these were the same subjects that were misclassified using our original logistic regression method, suggesting either a linear or non-linear multivariate modeling approach is appropriate.

Pathway Enrichment Analysis Identifies Enrichment for Neurodevelopment and ASD Targets

Analysis of the highest-confidence target mRNAs of the 14 miRNAs yielded an average of 555 predicted targets for each miRNAs (7,764 total predicted interactions). Approximately 10% of these were targeted by more than 1 of the miRNAs, yielding approximately 7,000 distinct mRNA interactions (Appendix A). This large number of interactions was then filtered based on the predicted strength of the miRNA-mRNA interaction, as reflected in the miRDB output to include only the top 20% of predicted targets for each miRNA (Appendix A). This identified 1,347 unique strongly-predicted mRNA interactions. The specific high-confidence mRNAs were then examined for evidence of functional enrichment using DAVID (version 6.7). A total of 1,247 of the high-confidence mRNA targets had functional annotation available. Using stringent settings (EASE score threshold set to 2.0, with Multiple Linkage Threshold set to 0.7, Similarity Threshold set to 0.45, and Final Group Membership set to 4) revealed 310 total cluster mappings. The top subnodes in the annotation clusters were then examined, revealing more than 2-fold enrichment of genes involved in Transcriptional Activation (present in 5 subnodes and 2 annotation clusters) and genes involved in Neuron Projection (51 genes) and Axon Projection (31 of the same genes).

We further probed the high-confidence target genes for relevance to ASD by comparing them with the 740 protein-coding genes in the Simons Foundation Autism Database (AutDB). Our high-confidence list of 1,347 mRNA targets contained 108 (14.6%) that overlapped the AutDB list (Appendix B). This represented a significant 2.2-fold enrichment for ASD-associated genes compared to the that expected by chance alone (Odds Ratio=2.40, 95th CI=1.94-2.97, Fisher's Exact p value=7.1e-12). Notable among these ASD-associated mRNA targets were Fragile X Mental Retardation Protein 1 (FMR1) and Forkhead Box Protein P2 (FOXP2).

The genes which mapped to the enriched DAVID clusters and the AutDB candidate genes were combined to indicate those target mRNAs that might be expected to have the most functional relevance for ASD. This indicated the most enhancement for AutDB genes that mapped to the Neuron Projection and Axon Projection subnodes, and also highlighted a small number of genes with apparent pleiotropic effects on multiple subnodes, including FOXP2 and FMR1 (Appendix C).

Target miRNAs in the Saliva are Widely and Highly Expressed in Human Brain

As a final examination of the potential brain-relevance of the miRNA targets we identified, we analysed RNA sequencing data on miRNAs across the developing human brain (4 months to 23 years) as deposited in the public domain by Ziats and Rennert [14]. This analysis yielded results for 13 of the 14 miRNAs we found altered in ASD saliva. Notably, all 13 miRNAs were expressed in multiple brain regions, including the cerebellum, dorsolateral prefrontal cortex (PFC), ventrolateral PFC, medial PFC, orbitofrontal PFC, and hippocampus throughout childhood. Moreover, nine of the miRNAs were expressed at high read levels (>1000) while 4 were expressed at relatively low read levels (<1000). No relationship was seen between expression level and either age or sex. However, five of the miRNAs varied in expression across brain regions (miR-7-5p, miR-27a-3p, miR-140-3p, miR-191-5p, and miR-2467-5p) with most differences occurring in the cerebellum versus the hippocampus.

The current diagnosis of ASD relies on parental questionnaires that are less than 50% specific and not valid until 18-24 months of age. This approach is inefficient in diagnosing children with ASD and enrolling them in early intervention services. The results of the present study suggest that addition of saliva-based biomarker testing could potentially lower the age of diagnosis and improve the specificity of screening, reducing the burden on referral services. We suggest that ideal biomarker candidates should be 1) expressed in the brain, 2) physiologically or functionally relevant to neurodevelopment, 3) easily measured from peripheral samples, and 4) differentially expressed in individuals with ASD. This study has identified a set of 14 miRNAs in the saliva that fit these criteria.

Examination of miRNA expression patterns across human brain development demonstrated that the miRNAs within our set of 14 biomarker candidates was consistently expressed in multiple brain areas throughout childhood. Moreover, functional pathway analysis of these miRNAs reveals enrichment of a gene network involved in neurodevelopment as well as genes associated with ASD according to the Simons Foundation Autism Database. Although it is beyond the scope of this report to discuss all of the overlapping target mRNAs, we do point out two notable ones: Fragile X Mental Retardation 1 (FMR1) and Forkhead Box Protein P2 (FOXP2). The FMR1 protein product is widely expressed in neurons, regulates synaptic translation through miRNA interactions, and is disrupted in Fragile X Syndrome, the most common inherited cause of intellectual disability. Approximately 40% of children with Fragile X Syndrome meet the criteria for ASD. In a similar fashion, FOXP2 was the first gene implicated in developmental speech and language disorders, and missense mutations of FOXP2 result in verbal apraxia, a hallmark of ASD. Both of these genes were present in more than one subnode, with FOXP2 highly represented in the Transcriptional Activation and Neuron Projection subnodes.

Of the 246 miRNA targets measured in the saliva of ASD and control children a set of 14 showed significant differences in abundance and was more than 95% accurate at predicting ASD diagnosis in a multivariate nonlinear logistic regression model developed in the full discovery data set. 100-fold cross-validation using Monte-Carlo simulations with masking of ⅓ of the samples revealed 87.5% specificity and 81% sensitivity, with an overall accuracy of 84.4%. Together, these findings indicate that miRNA profiling of the saliva has the potential to nearly double the overall specificity of the current “gold standard” M-CHAT screening method.

In the training set, all of the ASD subjects were correctly classified and only two control subjects were misclassified (subjects 204 and 205). However, these subjects did not display extreme variation in Vineland scores (both had composite scores of 113) or age (8 and 11 years old), although one subject did have a past history of speech therapy (subject 205). Their only notable medical findings were a diagnosis of asthma treated with albuterol as needed (subject 204) and the fact that both children were heavier and taller on average than the age-based percentiles of the children in the control group (weight percentiles 96 and 100 compared with a group mean of 78; height percentiles 92 and 97 compared with a group mean of 71). Examination of the records of the additional children who were misclassified during the cross-validation also failed to reveal any consistent pattern or association of medical or demographic variables.

A number of the salivary miRNAs that we identified as differentially expressed in children with ASD have been previously described in studies of post-mortem cerebellar cortex (miR-23a-3p, miR-27a-3p, miR-7-5p, and miR-140-3p), lymphoblastoid cell lines (miR-23a-3p, miR-30e-5p, miR-191-5p), and serum (miR-27a-3p, miR-30e-5p) of children with ASD. Thus, there are three miRNAs differentially regulated across three tissue types in children with ASD (miR-23a-3p, miR-27a-3p, and miR30e-5p). It is worth noting that miR-23a functions cooperatively with miR-27a to regulate cell proliferation and differentiation and the pair of miRNAs have been reported to be dysregulated in a number of human disease states, including ASD. Levels of miR-23a also fluctuate in response to CNS injuries like cerebral ischemia or temporal epilepsy, both of which can be associated with ASD. Thus, the dysregulation of miR-23a-3p may represent a pathophysiological hallmark of ASD.

The most robustly altered miRNA (miR-628-5p) in the present study has not been identified in previous ASD studies, although it is expressed in the human brain throughout postnatal development and has been implicated in CNS pathology. For example, analysis of miRNA expression in human gliomas showed significantly decreased expression of miR-628-5p. This contrasts with miR-628-5p expression in the saliva of ASD subjects, where it was significantly increased.

Aside from the sample size and cross-sectional nature of this pilot study, another limitation is the age of ASD and control subjects it describes (4-14 years) which are not representative of the target population in which ASD biomarkers would ideally be utilized (0-2 years). However, selecting a homogenous group of subjects with mild ASD (as measured by ADOS) that was well-established and diagnosed by a developmental specialist requires subjects with long-standing diagnoses. An additional consideration is the feasibility of saliva collection for screening children less than two years of age. We suggest that saliva is not only found in abundance during the period of teething (6 months to 18 months), but is also the most painless to collect. Future studies will be needed to assess the utility of the current miRNAs in predicting outcomes based on saliva samples from children in this age range.

The method of diagnosing whether a subject has an autism spectrum disorder of the present invention can also be employed to provide a second or confirmatory diagnostic for subjects who have previously been identified as being at risk of ASD using another diagnostic means, such means including but not limited to the Infant Toddler Checklist and M-CHAT, an evaluation of family history or genetic testing. It can also be used specifically with pre-verbal subjects where tests like the M-CHAT are not appropriate.

The novel aspect of this study is that it identifies a set of miRNAs in the saliva that are expressed in the brain, impact genes related to brain development and ASD, and are changed in a highly-specific manner in children with ASD. The specificity of this set of 14 miRNAs for a diagnosis of ASD is nearly twice that of the M-CHAT, the current gold standard used in ASD screening. Though copy number variants (CNVs) and single nucleotide polymorphisms (SNPs) are considered important genetic risk factors for ASD, they account for less than 30 percent of cases when considered in total and no single CNV explains more than 1 percent of ASD incidence. In comparison, epigenetic mechanisms such as miRNAs have the potential to alter coordinated networks of genes related to specific functional classes. An unexpected finding in the present study was the relationship of saliva miRNA levels with standard neurodevelopmental measures of adaptive behavior and the convergence of miRNAs targets on both neurodevelopmental processes and ASD candidate genes. This makes makes miRNAs such as miR-27a, miR-23a and miR-628-5p intriguing potential functional biomarkers for ASD. Prospectively validating these miRNA changes in a population of younger children with positive M-CHAT questionnaires and larger independent cohort replication samples could provide compelling evidence for the addition of miRNA biomarker screening to the diagnosis of ASD.

Differential Expression and Diagnostic Utility of miRNAs in Saliva of ASD Children.

FIG. 4A shows a Hierarchical cluster analysis of top 14 miRNAs. These miRNAs were differentially expressed in ASD children (subject code includes letter A) compared with Controls (subject code include letter C). Color indicates average Z-score of normalized abundance. A Euclidian distance metric was used with average cluster linkages for FIG. 4A. FIG. 4B shows a Partial Least Squares Discriminant Analysis (PLS-DA) of the top 14 miRNAs showed the general separation of subjects into two clusters, using only three eigenvector components that collectively accounted for 55% of the variance of the data set. FIG. 4C ROC-AUC analysis of the training data set indicated a very high level of performance in the logistic regression classification test (100% sensitivity, 90% specificity, with an area under the curve (AUC) value of 0.97).

Monte-Carlo Cross-Validation Analysis of Top 14 miRNAs.

The robustness of the 14 miRNA biomarkers was evaluated in stepwise fashion by determining their ability to correctly classify subjects using 100 iterations of a multivariate PLS-DA with 2, 3, 5, 7, 10, and 14 miRNAs included, and masking of ⅓ of the subjects during the training phase. This revealed an overall ROC-AUC of 0.92 and mis-classification of 3 ASD and 4 Controls.

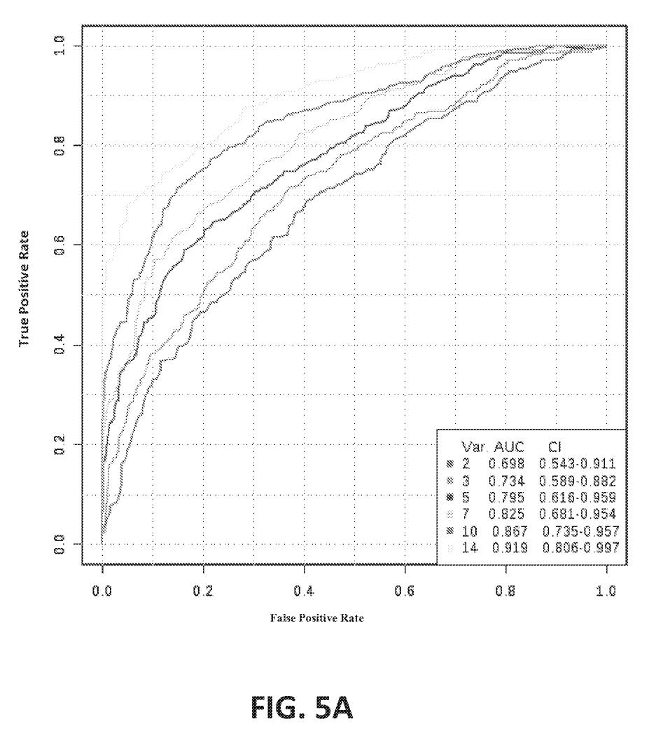

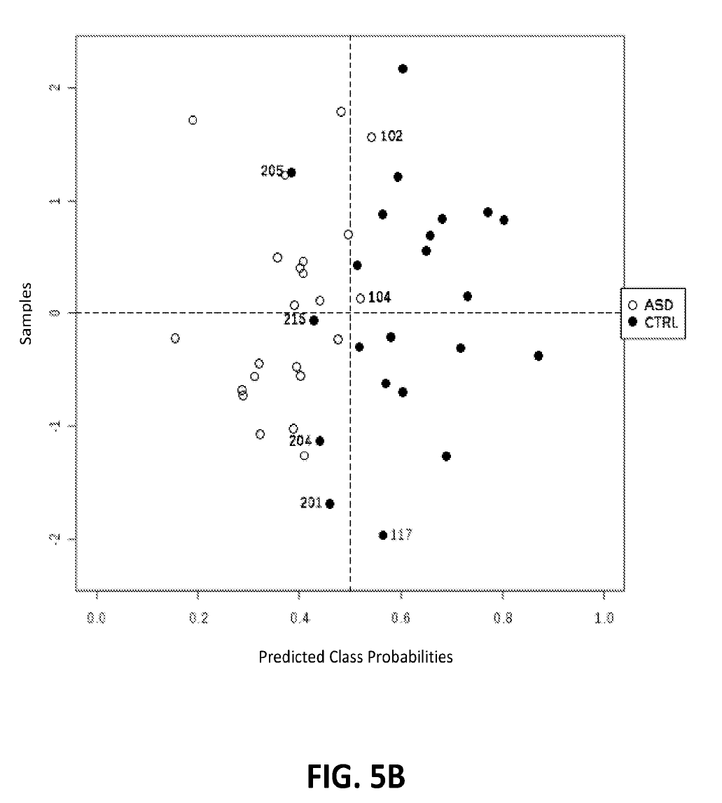

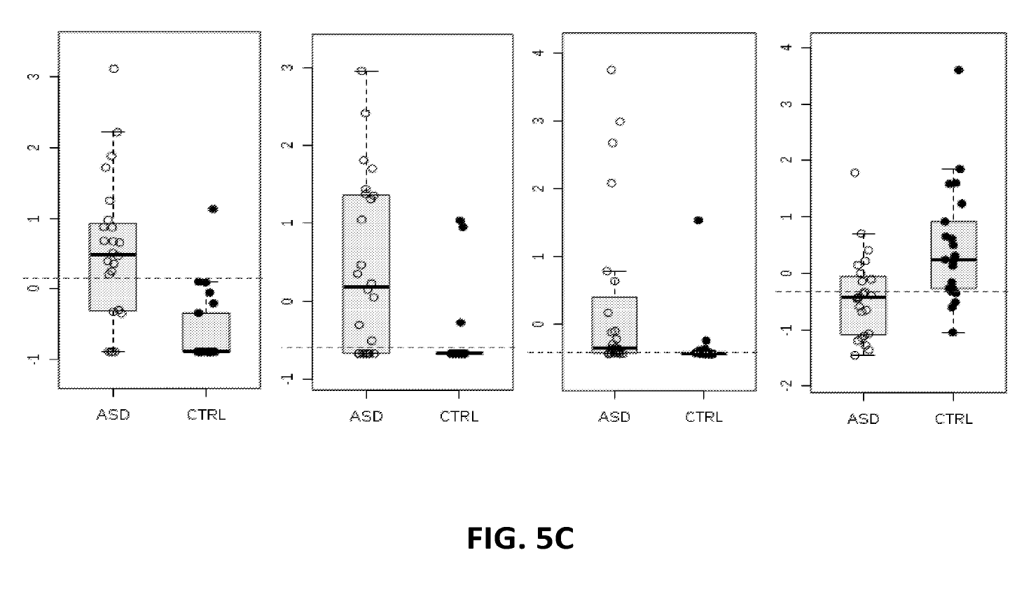

FIG. 5A shows the robustness of the 14 miRNA biomarkers was evaluated in stepwise fashion by determining their ability to correctly classify subjects using 100 iterations of a multivariate PLS-DA with 2, 3, 5, 7, 10, and 14 miRNAs included, and masking of ⅓ of the subjects during the training phase. This revealed an overall ROC-AUC of 0.92 and mis-classification of 3 ASD and 4 Controls. FIG. 5B shows the classification of subjects plotted by probabilities from the MCCV, with incorrectly classified subjects identified by ID number. FIG. 5C shows a Whisker box plots of the four most robustly changed miRNAs according to the Mann-Whitney test.

What is claimed is:

1. A collection of 2 or more miRNA probes of a probe set having ribonucleotide sequences selected from the group comprising SEQ ID NOs. 1, 2, 3, 4, 5, 6, 7, 8, 9, 10, 11, 12, 13, and 14.

2. The collection of claim 1, wherein the collection comprises a relative ratio of 1 or more of the 14 miRNA probes to miRNAs present in a biological sample.

3. A miRNA microarray comprising:
a solid support and
the collection of claim 1, wherein the collection is attached to the solid support.

4. A method of diagnosing whether a subject has an autism spectrum disorder, said method comprising:
obtaining a biological sample from a subject potentially having an autism spectrum disorder;
providing a collection of 1 or more miRNA probes of a probe set having a ribonucleotide sequence selected from the group comprising SEQ ID NOs. 1, 2, 3, 4, 5, 6, 7, 8, 9, 10, 11, 12, 13, and 14;
contacting the biological sample from the subject with said collection under conditions effective to permit hybridization of said probes to complementary nucleic acid molecules, if present, in the biological sample;
detecting any hybridization as a result of said contacting; and
identifying whether the subject has an autism spectrum disorder based on said detecting.

5. The method of claim 4, further comprising:
effectuating a therapy guidance based on said identifying.

6. The method according to claim 5, wherein the therapy guidance involves one or more of the following: performing additional diagnostic testing, prescribing a drug therapy, increasing monitoring frequency of the subject's autism spectrum disorder, recommending behavioral therapy and lifestyle choices, or a combination thereof.

7. The method according to claim 6, wherein the therapy guidance involves recommending lifestyle choices and includes one or more of the following: introducing behavioral techniques, managing time and task organization, introducing environmental changes, changes in diet, changes in exercise, or a combination thereof.

8. The method of claim 4, wherein the collection comprises relative ratios of 1 or more of the 14 miRNA probes to other miRNAs present in the sample.

9. The method of claim 4, wherein the biological sample is saliva, urine, stool, serum, plasma, brain tissue, or cerebrospinal fluid.

10. The method of claim 4, wherein said detecting is carried out by quantitative hybridization-based assays, including microarrays, Luminex-based magnetic or non-magnetic beads, Northern blot, RNase-Protection Assay, in situ hybridization (ISH), RNA-Scope and SMART Flare.

11. The method of claim 4, wherein said detecting determines miRNA expression level.

12. The method of claim 4, wherein said detecting is carried out by a polymerase chain reaction assay, including real-time quantitative PCR with oligonucleotide probes, SybrGreen or similar dye, radioactive nucleotide-based quantification, as well as digital droplet PCR.

13. A method of diagnosing whether a subject has an autism spectrum disorder, said method comprising:
obtaining a biological sample from a subject potentially having an autism spectrum disorder;
subjecting the biological sample to a direct sequencing process to provide nucleotide sequence information for nucleic acid molecules in the biological sample;
comparing the nucleotide sequence information for nucleic acid molecules in the biological sample to nucleotide sequences of a collection of 1 or more miRNA probes selected from the probe set comprising SEQ ID NOs. 1, 2, 3, 4, 5, 6, 7, 8, 9, 10, 11, 12, 13, and 14;
determining whether any of the collection of 1 or more miRNA probes or their complements are present in the biological sample, and determination of their level of expression, and relative levels of expression to each other based on said comparing; and
identifying whether the subject has an autism spectrum disorder based on said determining.

14. The method of claim 13, further comprising:
effectuating a therapy guidance based on said identifying.

15. The method according to claim 14, wherein the therapy guidance involves one or more of the following: performing additional diagnostic testing, prescribing a drug therapy, increasing monitoring frequency of the subject's condition, recommending behavioral therapy and lifestyle choices, or a combination thereof.

16. The method according to claim 14, wherein the therapy guidance involves recommending lifestyle choices and includes one or more of the following: introducing behavioral techniques, managing time and task organization, introducing environmental changes, changes in diet, changes in exercise, or a combination thereof.

17. The method of claim 13, wherein the collection comprises relative ratios of 1 or more of the 14 miRNA probes to other miRNAs present in the sample.

18. The method of claim 13, wherein the biological sample is saliva, urine, stool, serum, brain tissue, or cerebrospinal fluid.

19. A kit suitable for determining whether a subject has an autism spectrum disorder, said kit comprising:
2 or more miRNA probes of a probe set having ribonucleotide sequences selected from the group comprising SEQ ID NOs. 1, 2, 3, 4, 5, 6, 7, 8, 9, 10, 11, 12, 13, and 14.

20. The kit of claim 19, further comprising a solid support attached to said 2 or more miRNA probes.

21. The kit of claim 19, further comprising:
at least one of the following: (a) one randomly-generated sequence used as a negative control; (b) at least one oligonucleotide sequence derived from a housekeeping gene, used as a standardized control for total RNA degradation; or (c) at least one randomly-generated sequence used as a positive control.

22. A method of treating autism spectrum disorder in a subject, the method comprising:
obtaining a biological sample from the subject;
providing a collection of 1 or more miRNA probes of a probe set having a ribonucleotide sequence selected from the group comprising SEQ ID NOs. 1, 2, 3, 4, 5, 6, 7, 8, 9, 10, 11, 12, 13, and 14;
contacting the biological sample with said collection under conditions effective to permit hybridization of said probes to complementary nucleic acid molecules, if present, in the biological sample;
detecting any hybridization as a result of said contacting;
identifying whether the subject has an autism spectrum disorder based on said detecting; and
effectuating a therapy guidance based on said identifying.

23. A method of treating autism spectrum disorder in a subject, the method comprising:
obtaining a biological sample from a subject potentially having an autism spectrum disorder;
subjecting the biological sample to a direct sequencing process to provide nucleotide sequence information for nucleic acid molecules in the biological sample;
comparing the nucleotide sequence information for nucleic acid molecules in the biological sample to nucleotide sequences of a collection of 1 or more miRNA probes selected from the probe set comprising SEQ ID NOs. 1, 2, 3, 4, 5, 6, 7, 8, 9, 10, 11, 12, 13, and 14;
determining whether any of the collection of 1 or more miRNA probes or their complements are present in the biological sample, and determination of their level of expression, and relative levels of expression to each other based on said comparing;
identifying whether the subject has an autism spectrum disorder based on said determining; and
effectuating a therapy guidance based on said identifying.

24. The microarray of claim 3, wherein the solid support comprises a swab, saliva collection vial, and/or a PCR array plate.

25. The kit of claim 20, wherein the solid support comprises a swab, saliva collection vial, and/or a PCR array plate.

26. The collection of claim 2, wherein the relative ratio is between 1.5:1 and 2.5:1.

27. The method of claim 8, wherein the relative ratios are between 1.5:1 and 2.5:1.

28. The method of claim 17, wherein the relative ratios are between 1.5:1 and 2.5:1.
